(19) United States
(12) Patent Application Publication
(10) Pub. No.: US 2016/0174986 A1
(43) Pub. Date: Jun. 23, 2016

(54) ANASTOMOSIS DEVICES AND METHODS OF USING SAME

(71) Applicant: THE CLEVELAND CLINIC FOUNDATION, Cleveland, OH (US)

(72) Inventors: David G. Vince, Westlake, OH (US); Grant T. Hoffman, Terre Haute, IN (US)

(21) Appl. No.: 14/790,584

(22) Filed: Jul. 2, 2015

Related U.S. Application Data
(60) Provisional application No. 62/020,063, filed on Jul. 2, 2014.

(51) Int. Cl. A61B 17/11

(52) U.S. Cl. CPC A61B 17/11

(57) ABSTRACT

A device and method for supporting a joint formed by first and second patient tissue structures are provided. A device body includes a band having a first band end and a second band end laterally separated by a band length. The first and second band ends are configured for selective mutual interconnection. A top band portion and a bottom band portion are longitudinally separated by a band height. An inner surface of a device body is directly adjacent to the joint and is attached to at least one of the first and second patient tissue structures during use. An outer surface of the device body, oppositely facing from the inner surface, includes a semi-permeable membrane. The membrane permits liquid to penetrate therethrough in a first direction from the outer toward the inner surface. The membrane substantially prevents liquid from penetrating therethrough in a second direction opposite the first direction.

### RELATED APPLICATION

This application claims priority from U.S. Provisional Application No. 62/020,063, filed 2 Jul. 2014, the subject matter of which is incorporated herein by reference in its entirety.

### TECHNICAL FIELD

The present invention relates to devices and methods for sealing an anastomosis site to prevent leakage of biological fluid from the site.

### BACKGROUND

An anastomosis is a surgical connection between at least two bodily structures. Usually, the connection is created between two tubular or luminal structures. An anastomosis can be performed on blood vessels, the gastrointestinal (“GI”) and digestive tract, the urinary tract, the reproductive tract, and other parts of the body. A “reanastomosis” is a surgical reconnection usually reversing a prior surgery to disconnect an anatomical anastomosis. A reanastomosis can be used, for example, to restore fertility after tubal ligation or a vasectomy.

Certain surgical procedures, including a vascular bypass operation (e.g., a coronary artery bypass), an aneurysmectomy, and a solid organ transplant, generally require vascular anastomoses. An anastomosis connecting an artery to a vein is also used to create an arteriovenous fistula as an access point for hemodialysis. Regarding GI and digestive tract anastomosis, virtually all elective resections of gastrointestinal organs are followed by anastomoses to restore continuity. Bypass operations on the GI or digestive tract, once rarely performed, are now the cornerstone of bariatric surgery. Another type of anastomosis of the digestive tract, colonic anastomosis, is used to restore colonic continuity after resection of the colon, such as during surgical treatment for cancer. Anastomoses of parts of the urinary tract include radical prostatectomy (“RP”) and radical cystectomy, which are performed for prostate cancer. These procedures involve anastomosis of the bladder to the urethra (vesicourethral anastomosis or “VUA”) after the prostate is removed to restore continuity of the urinary tract.

Depending on the circumstances, a surgical anastomosis can be created using sutures sewn manually or robotically, mechanical staplers, or biological glues. Suturing is difficult to do precisely and may be time consuming when done by the robotic approach. Further, sutures may tear through tissue resulting in undesired extravasation. Sutures may also predispose the tissue to strictures, the treatment of which may require additional surgical procedures to correct, thus increasing the risk of complications.

### SUMMARY

In an aspect, a device for supporting a joint formed by adjacent first and second patient tissue structures is provided. The device includes an inner surface of a device body. The inner surface is directly adjacent to the joint and is attached to at least one of the first and second patient tissue structures during use. An outer surface of the device body, oppositely facing from the inner surface, is provided. The outer surface includes a semi-permeable membrane. The membrane permits liquid to penetrate therethrough in a first direction from the outer toward the inner surface. The membrane substantially prevents liquid from penetrating therethrough in a second direction opposite the first direction.

In an aspect, a method of supporting a joint formed by adjacent first and second patient tissue structures is provided. A device is provided, the device including an inner surface of a device body. The device includes an outer surface of the device body, oppositely facing from the inner surface. The outer surface includes a semi-permeable membrane. The device is removably attached to a delivery device. The device is manipulated with the delivery device into proximity with the joint to establish an operative configuration such that the inner surface is directly adjacent to the joint in a supporting relationship and the inner surface is attached to at least one of the first and second patient tissue structures. The device is maintained in the operative configuration. Liquid is permitted to penetrate through the membrane in a first direction from the outer toward the inner surface. Liquid is substantially prevented from penetrating through the membrane in a second direction opposite the first direction.

In an aspect, a device for supporting a joint formed by adjacent first and second patient tissue structures is provided. A device body includes a band having a first band end and a second band end laterally separated by a band length. The first and second band ends are configured for selective mutual interconnection. A top band portion and a bottom band portion are longitudinally separated by a band height. An inner surface of a device body is directly adjacent to the joint and is attached to at least one of the first and second patient tissue structures during use. An outer surface of the device body, oppositely facing from the inner surface, includes a semi-permeable membrane. The membrane permits liquid to penetrate therethrough in a first direction from the outer toward the inner surface. The membrane substantially prevents liquid from penetrating therethrough in a second direction opposite the first direction.

### BRIEF DESCRIPTION OF THE DRAWINGS

For a better understanding, reference may be made to the accompanying drawings, in which:

FIG. 1 is a schematic plan view of an embodiment of a device;

FIG. 2 is a schematic side view of the embodiment of FIG. 1;

FIG. 3 is a schematic side view of the embodiment of FIG. 1 in an example use environment;

FIG. 4 is a schematic plan view of an embodiment of the device in a first arrangement;

FIG. 5 is a schematic plan view of the embodiment of FIG. 4 in a second arrangement;

FIG. 6 is a perspective view of the embodiment of FIG. 5 in an operative configuration;

FIG. 7 is a perspective view of the embodiment of FIG. 6 depicting an optional component in the operative configuration;

FIG. 8 is a perspective view of the embodiment of FIG. 6 in the example use environment; and

FIGS. 9A-9C depict an example sequence of use of the embodiment of FIG. 4.

### DESCRIPTION OF ASPECTS OF THE DISCLOSURE

Unless defined otherwise, all technical and scientific terms used herein have the same meaning as is commonly understood by one of skill in the art to which the present disclosure pertains.

The invention comprises, consists of, or consists essentially of the following features, in any combination.

The present invention relates to anastomosis devices and methods to seal a surgical anastomosis site. Although the disclosure herein refers primarily to a vesicourethral anastomosis (hereafter, a “VUA”), the devices and methods can be used to anastomose other bodily structures of a patient in need thereof. As used herein, the term “subject” can be used interchangeably with the term “patient”, either of which can refer to any warm-blooded organism including, but not limited to, human beings, pigs, rats, mice, dogs, goats, sheep, horses, monkeys, apes, rabbits, cattle, farm animals, livestock, etc. As used herein, a “patient” includes a mammal, preferably a human being. The disclosure herein also refers to the term “substantially” with respect to certain geometric shapes. By “substantially”, it is meant that the shape of the element need not have the mathematically exact described shape but can have a shape that is recognizable by one skilled in the art as generally or approximately having the described shape. Also, the disclosure herein refers to an “operative configuration.” An “operative configuration” refers to the configuration of a device when it is deployed in the body. The disclosure also refers to the terms “top portion” and “bottom portion.” These terms do not necessarily refer to the position of a device in an operative configuration when a patient is in a standard anatomical position. Rather, these terms are used to distinguish opposing portions of the device irrespective of their actual orientation in a patient's body in an operative configuration.

Further, as used herein with respect to a described element, the terms “a,” “an,” and “the” include at least one or more of the described element unless otherwise indicated. Further, the term “or” refers to “and/or” unless otherwise indicated. In addition, it will be understood that when an element is referred to as being “on,” “attached” to, “connected” to, “coupled” with, “contacting,” in “communication” with etc., another element, it can be directly on, attached to, connected to, coupled with, contacting, or in communication with the other element or intervening elements may also be present. In contrast, when an element is referred to as being, for example, “directly on,” “directly attached” to, “directly connected” to, “directly coupled” with, “directly contacting,” or in “direct communication” with another element, there are no intervening elements present. It will also be appreciated by those of skill in the art that references to an element that is disposed “adjacent” another element may have portions that overlap or underlie the adjacent element.

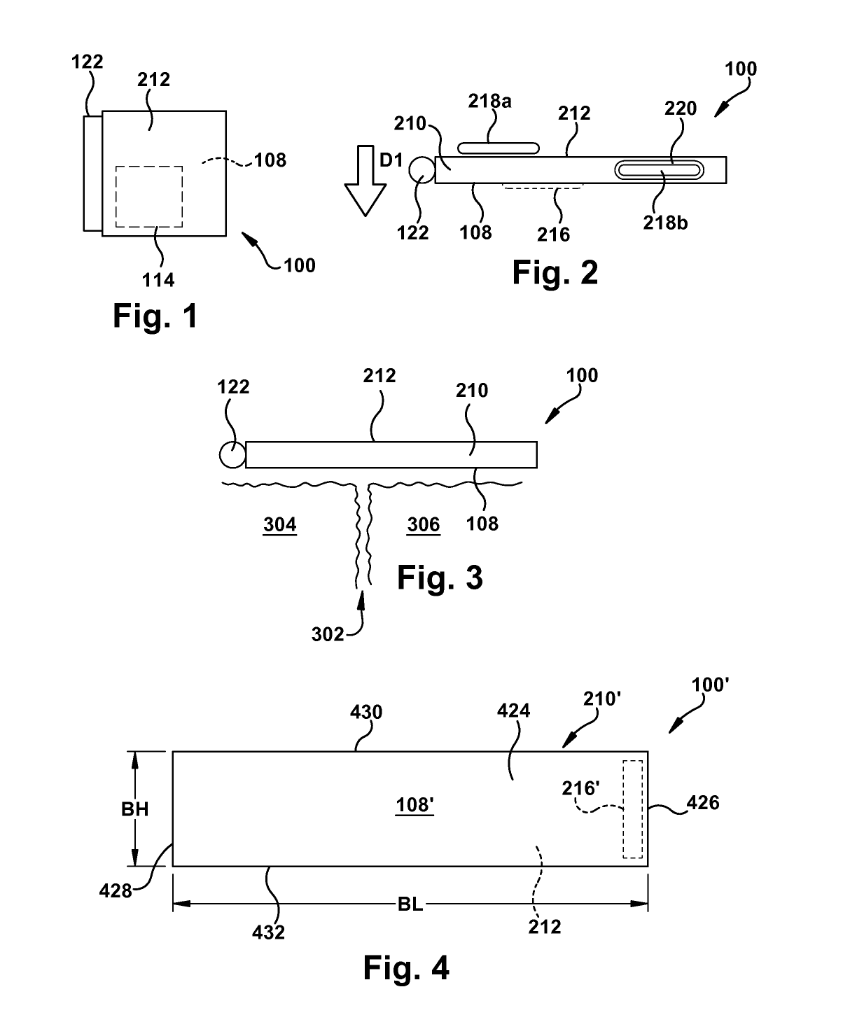

FIGS. 1-3 depict a device 100 for supporting a joint 302 formed by adjacent first and second patient tissue structures 304 and 306, respectively. The device 100 includes an inner surface 108 of a device body 210. The inner surface 108 is, in use, directly adjacent to the joint 302. The inner surface 108 is attached to at least one of the first and second patient tissue structures 304 and 306 during use, although the inner surface 108 is shown slightly separated from these structures in the schematic view of FIG. 3, for clarity of depiction. Optionally, the inner surface 108 may directly contact (whether or not attached thereto) one or both of the first and second patient tissue structures 304 and 306 during use.

The device 100 may be used to at least partially seal the joint 302. The term “seal” is used herein to indicate that the device 100 substantially prevents passage of fluid through the joint 302 at the “sealed” portion. This sealing action can occur through the device 100 directly blocking passage of fluid through the joint 302, the device 100 approximating the first and second patient tissue structures 304 and 306 to indirectly block passage of fluid through the joint 302, or in any other suitable manner. For many anatomical joints 302 formed by first and second patient tissue structures 304 and 306, blood will naturally coagulate and assist the device 100 to seal the joint 302. However, in use environments where the fluid potentially flowing through the joint 302 is noncoagulatory (e.g., urine), the device 100 may take on more of an active sealing role than in some other, more blood-containing use environments where the blood will assist with the sealing process.

The device 100, or components thereof, can biodegrade over time, can be removed from the joint 302 after a predetermined amount of healing of the joint 302 has taken place, or can permanently remain within the patient's body. If the device 100 is at least partially intended to biodegrade, then it is contemplated that at least that biodegradable portion of the device 100 may be made from a biodegradable material. However, other suitable materials which may be used to at least partially form the device 100 include, but are not limited to, polyetheretherketone (PEEK); ultra-high-molecular weight-polyethylene (UHMWP); polyurethanes and composites, including composites containing carbon fiber; natural or synthetic elastomers such as polyisoprene (natural rubber); biodegradable materials such as Poly-lactic acid (PLA) and its isomers and copolymers, Poly-glycolic acid (PGA), Poly-caprolactone (PCL), Poly(dioxanone), Poly-lactide-co-glycolide, and biodegradable Magnesium alloys using Mg, Zn, Li, Al, Ca and rare earths; synthetic polymers and copolymers; thermoplastic elastomers such as polyurethane elastomers, including polycarbonate-urethane elastomers; and any suitable combination thereof.

The device 100 also includes an outer surface 212 of the device body 210. The outer surface 212 is oppositely facing from the inner surface 108, as shown in FIG. 2. The device body 210 could be a separate structure from the inner and/or outer surfaces 108 and 212, or one or both of the inner and outer surfaces 108 and 212 could itself form at least a portion of the device body 210.

In a very simple configuration, for example, the inner and outer surfaces 108 and 212 could merely be the obverse and reverse of a single sheet of material, with the extremely thin “layer” of sheet volume between those two surfaces serving as the device body 210. As a more complex example, the inner and outer surfaces 108 and 212 could be outwardly-facing surfaces of two different sheets of material, with the inwardly-facing surfaces of those sheets comprising the device body 210. Yet another example of a suitable device 100 construction includes inner and outer surfaces 108 and 212, which are outwardly-facing surfaces of two different sheets of material, with at least one separate “device body” sheet of material laminated or sandwiched therebetween. One of ordinary skill in the art can provide a device 100 structure suitable for a desired use environment according to the teachings herein.

The outer surface 212 includes a semi-permeable membrane (shown schematically by the dashed line 114 in FIG. 1) forming part or all of the outer surface 212. The membrane 114 permits liquid to penetrate therethrough in a first direction (arrow D1 in FIG. 2) from the outer surface 212 toward the inner surface 108. However, the membrane 114 substantially prevents liquid from penetrating therethrough in a second direction, opposite the first direction D1. The membrane 114 could be at least partially made, for example, from a sheet (such as a knitted or woven fabric) of expandable polytetrafluoroethylene (ePTFE), PTFE, polyester (for example, Dacron® polyethylene terephthalate), polyethylene, polypropylene, Poly-lactic acid (PLA) and its isomers and copolymers, Poly-glycolic acid (PGA), Poly-caprolactone (PCL), Poly(dioxanone), Poly-lactide-co-glycolide any other desired materials, or any suitable combination thereof. The membrane 114 could comprise part or all of the outer surface 212. The membrane 114 could be a separate component from the outer surface 212 or could be at least partially formed integrally therewith.

At least a portion of the inner surface 108 may include an adhesive, shown schematically as dotted line 216 in FIG. 2. The adhesive 216 may be used to attach at least a portion of the inner surface 108 to one or both of the first and second patient tissue structures 304 and 306, to another structure of the device 100, as discussed below, or for any other reason. The adhesive 216 may be of any desired type, though will likely be biocompatible for most patient tissue use environments of the device 100. For example, the adhesive 216 may be thermally, chemically, or photo-activated by any suitable activator, such as at least one of a thermally-activated bioadhesive, a chemically-activated bioadhesive, and a photo-activated bioadhesive.

For example, the adhesive can be a hydrogel glue. One example of a suitable activator for some adhesives is hydrogen peroxide, which can be delivered directly by a syringe, for example, to the inner surface 108 to harden the adhesive. For some use environments and types of adhesive, a liquid activator may be permitted to permeate by the membrane 114 to pass through the outer surface 212 and at least a portion of the device body 210 to activate the adhesive 216 from “below” the inner surface 108 and thus adhere the inner surface 108 to other portions of the device 100 and/or to the joint 302, the first patient tissue structure 304, and/or the second patient tissue structure 306. For example, the adhesive 216 could at least partially attach the inner surface 108 to at least one of the first and second patient tissue structures 304 and 306 during use of the device 100.

Optionally, at least one reinforcement feature 218 could be provided to locally resist force applied against the device 100 in substantially at least one of the first (D1) and second directions. For example, the reinforcement feature(s) 218 could assist with maintaining the device 100 in an initial (e.g., substantially planar, as shown in FIGS. 1-3) configuration. The reinforcement feature 218 could be provided in any suitable manner. One suitable reinforcement feature 218 could be an integral (i.e., not separately provided) feature resulting from a thickness of a local portion of the device body 210 or some other portion of the device 100—a “rib” or other locally thickened portion of the device 100 could act as a stiffener.

Another example of a reinforcement feature 218 is a separate strut provided to the device 100. Two types of struts are shown in FIG. 2 in schematic side view. Reinforcement feature 218a is a strut attached to the outer surface 212. Reinforcement feature 218b is a strut encapsulated within the device body 210, such as by being molded or otherwise formed into the device body 210 and/or being inserted into a pocket 220 formed in the device body 210.

The reinforcement feature(s) 218, when present and of the “separate strut” variety, may be made from materials including, but not limited to, plastically or elastically deformable polymers and metals. Example plastics include polyetheretherketone (PEEK); ultra-high-molecular weight-polyethylene (UHMWP); polyurethanes and composites, including composites containing carbon fiber; natural or synthetic elastomers such as polyisoprene (natural rubber); synthetic polymers and copolymers; thermoplastic elastomers such as polyurethane elastomers, including polycarbonate-urethane elastomers; or any suitable combination thereof. An example metal is NiTinol.

The device 100 of FIGS. 1-3 could be delivered to the site of the joint 302 in any desirable manner. For example, a coupling feature (shown schematically at 122) could be selectively attachable to a delivery device (not shown) to aid placement of the device 100 at and/or near the joint 302, in any desired manner. Once the device 100 has achieved the operative configuration, as shown in FIG. 3, the device 100 helps to support and/or seal the joint 302 as desired.

A device 100 can be used to connect body structures together including vascular lumens, non-vascular lumens, and other tubular bodily structures. Non-limiting examples of such lumens and tubular bodily structures include vascular structures such as veins and arteries; portions of the gastrointestinal and digestive tract (including, for example, use in gastric bypass surgery) including the esophagus, stomach, small intestine, large intestine, colon, bile ducts, and pancreas; the urinary tract, including the ureters, urinary bladder, and urethra; the reproductive tract, including the fallopian tubes and the vas deferens. These listed bodily structures are only provided as examples, and other suitable bodily structures (e.g., substantially planar or curvilinear structures) could be supported and/or sealed using the device 100. In certain embodiments, the device 100 may be used in applications where fluid does not coagulate, such as urine. For example, the device 100 can be used to help support and/or seal a sutured VUA following a radical prostatectomy or radical cystectomy. A device 100 can also be used for reanastomosis, such as to restore fertility, for example, after tubal ligation or a vasectomy.

FIGS. 4-9 depict a device 100′ in a second configuration. The device 100′ of FIGS. 4-9 is similar to the device 100 of FIGS. 1-3 and therefore, structures of FIGS. 4-9 that are the same as or similar to those described with reference to FIGS. 1-3 have the same reference numbers with the addition of a “prime” mark. Description of common elements and operation similar to those in the previously described first configuration will not be repeated with respect to the second configuration.

In the second configuration, as shown in FIG. 4, the device body 210′ includes a band 424 having a first band end 426 and a second band end 428 laterally separated by a band length BL. The first and second band ends 426 and 428 (and/or adjacent portions of the band 424) are configured for selective mutual interconnection. The term “interconnection” is used here to indicate a situation where structures are or become mutually connected. The band 424 also includes a top band portion 430 and a bottom band portion 432 longitudinally separated by a band height BH.

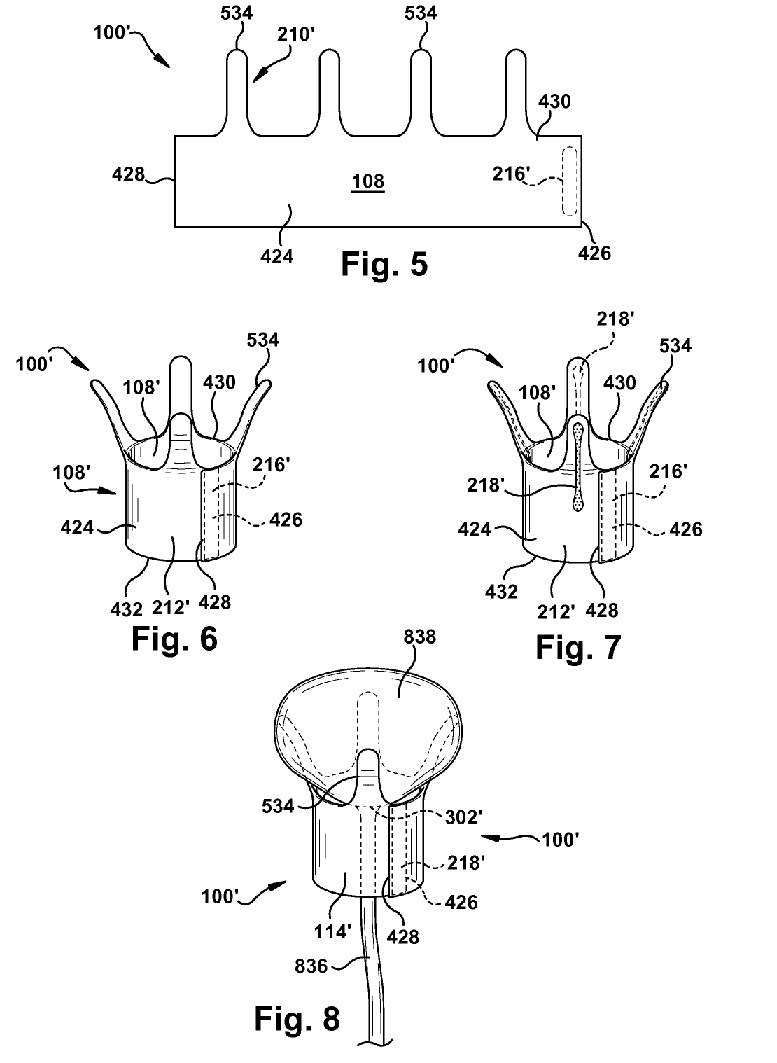

Optionally, and as shown in FIG. 5, the device body 210′ may include a plurality of fingers 534 extending substantially longitudinally from the top band portion 430 in a direction away from the bottom band portion 432.

The second embodiment of the device 100′ is described herein for convenience as supporting and/or sealing a VUA to connect a bladder with a urethra, but the device 100′ can be used for other types of anastomosis or for any other purpose as desired. When the device 100′ includes a plurality of fingers 534, the plurality of fingers 534 can be any suitable number of fingers 534 for supporting and/or sealing the first and/or second patient tissue structures 304′ and 306′ that are anastomized at the joint 302′. The number of fingers 534 provided will at least partially be provided responsive to the particular use environment and anastomized body structures. However, in the case of a VUA, there will often be three or more fingers 534. Further, in the case of a VUA, the fingers 534 may have a length of, for example, between about one centimeter (cm) and about three cm for many patients, but the fingers 534 could have shorter or longer lengths, as desired. In the case of the vas deferens, the fingers 534, if any, can have a length of about 1 millimeter (mm). Regarding the length of the fingers 534, it is contemplated that a user could trim previously configured fingers 534, to any desired length(s), tip profile(s), or any other dimensions as desired for a particular use environment, including a combination of various dimensions among the plurality of fingers 534.

Optionally, the material of the fingers 534 may allow the fingers to elastically or plastically deform. In embodiments where a finger 534 of the device 100′ is deformable, the entire finger 534, or only a portion thereof, may be fabricated from a deformable material, to allow part or all of the finger 534 to deform and better support and/or seal one or more of the first and second patient tissue structures 304 and 306.

This situation, with deformable fingers 534, is shown in FIGS. 6-8. In these Figures, the device 100′ is shown in an operative configuration. The band 424 is shown as having been rolled into a substantially cylindrical/tubular/loop-like configuration, though can be connected in any suitable manner and to form any desired structure for a particular use environment. The first and second band ends 426 and 428 are interconnected to maintain the band in the operative configuration, here at least partially via circumferential overlapping of the first and second band ends 426 and 428 and activation of the adhesive 218′ to maintain the device 100′ in the operative configuration.

The presence of the (optional) fingers 534 in FIGS. 6-8 imparts a crown-like configuration to the device 100′ in the operative configuration. The term “crown-like” is used herein to indicate a structure having a circumferentially extending loop (here, the substantially cylindrical structure formed by the joined-end band 424) with at least one protrustion/extension (e.g., the fingers 534) extending therefrom, as shown in FIGS. 6-8. Optionally, and also as shown in FIGS. 6-8, the fingers 534 may flare outward from the top band portion 430, as desired for a particular use environment.

Optionally one or more reinforcement features 218′ may be provided at least partially to the fingers 534, perhaps extending from the fingers 534 down toward the band 424. The reinforcement feature(s) 218′, when present, may serve to locally resist force applied against at least one of the plurality of fingers 534 in substantially at least one of the first (outward from the inner surface 108′) and second (opposite the first) directions. As with the previously discussed reinforcement features 218 of the first embodiment, the reinforcement features 218′ of the second embodiment can be an integral feature or a separate strut. In the latter situation, whether the reinforcement feature 218′ is attached to the outer surface 212′ or encapsulated within the device body 210′ , the reinforcement feature 218′ may be attached to one or more of: (1) at least a portion of a finger 534, and (2) at least a portion of the band 424. The reinforcement features 218′ may allow the fingers 534 to elastically or plastically deform and exert forces as desired.

Whether or not reinforcing features 218′ are provided, the fingers 534, and the device 100′ may be useful to help support and/or seal a VUA (joint 302′) between a urethra 836 and a bladder 838. (Optionally, the urethra 836 and bladder 838 may have already been attached, such as via surgical adhesives, sutures, staples, and/or any suitable fasteners or combinations thereof, to create the VUA. Alternately, the device 100′ could be used to help anastomose the urethra 836 and bladder 838 directly) For example, and as shown in FIG. 8, the device 100′ may be manipulated, in any suitable manner, into proximity with the joint 302′ to establish an operative configuration, such as with the crown-like configuration depicted. In this operative configuration, the inner surface 108′ may be directly adjacent to the joint 302′ in a supporting relationship, and the inner surface 108′ may be attached to at least one of the first and second patient tissue structures 304′ and 306′ (here, the urethra 836 and bladder 838, respectively). When the inner surface 108′ includes an appropriately positioned adhesive 218′, the inner surface 108′ could be adhered to at least one of the first and second patient tissue structures 304′ and 306′ (here, the urethra 836 and bladder 838, respectively). The device 100′ could also be attached to the urethra 836 and/or the bladder 838 using one or more of surgical adhesives, sutures, staples, and/or any suitable fasteners.

Also in the operative configuration, the first and second band ends 426 and 428 are connected to form a circumferentially extending loop encircling at least a portion of the joint 302′. Again, when the inner surface 108′ includes an appropriately positioned adhesive 218′, an activator (not shown) could be allowed to permeate through the membrane 114′ to activate the adhesive 218′ and thus connect the first and second band ends 426 and 428 into the crown-like, encircling-loop configuration shown.

The device 100′ could fit as closely as desired to the various patient tissue structures associated with the joint 302. For example, and as shown, the fingers 534 could be used to help brace and support the bladder 838 to ease or prevent undue stressing forces upon the VUA joint 302′ while the joint heals. The device 100′ could also serve to at least partially seal the joint 302′. In FIG. 8, since urine will not clot or coagulate as blood would, it may be especially helpful to have sealing assistance for a VUA joint 302′.

The device 100′ may remain in place relative to the joint 302′ and first/second patient tissue structures 304′/306′ for a desired period of time, to physically help support and/or seal these patient body structures during the healing process. Optionally, at least part of the device 100′ may biodegrade, but it is also contemplated that a surgical procedure could be performed to remove at least a portion of the device 100′ from the site at a desired time after the initial installation, even if the device 100′ is at least partially biodegradable.

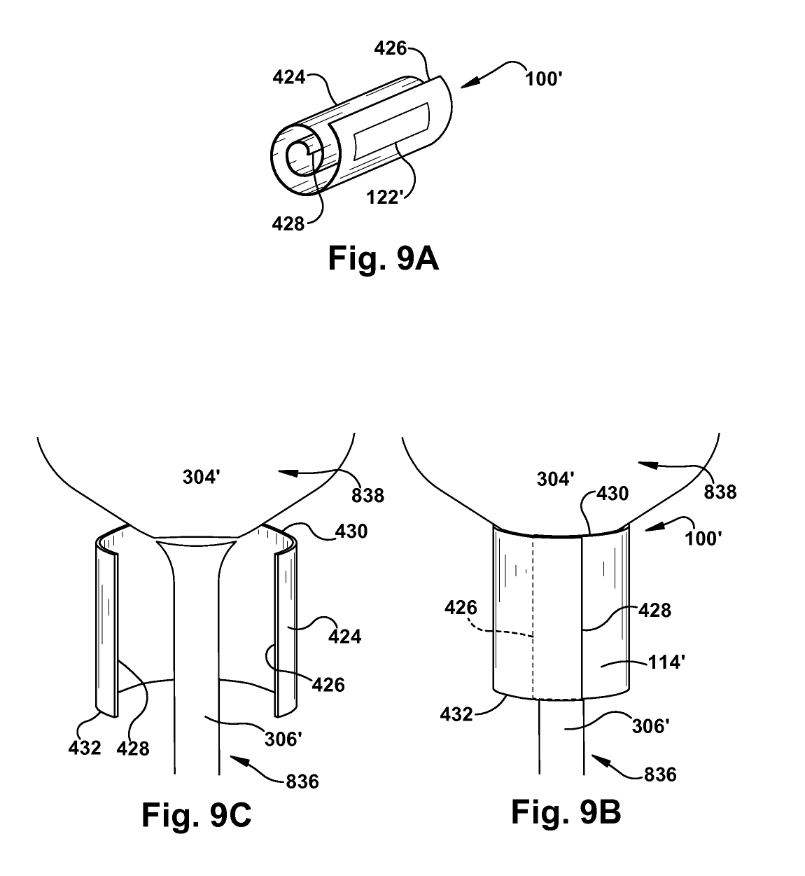

FIGS. 9A-9C schematically depict the installation procedure for a device 100′ without fingers 534 for clarity, though any device 100′ could be similarly installed. Here, the device 100′ includes a coupling feature 122′ selectively attaching to a delivery device (e.g., a catheter, endoscope, trocar, stylet, forceps, or any other suitable delivery device, not shown) to aid placement of the device 100′ at and/or near the joint 302. As this point, the device 100′ is delivered to the anastomosis site as a substantially flat band 424 with free first and second band ends 426 and 428, which is rolled or otherwise collapsed for delivery, as shown in FIG. 9A.

Once the device 100′ is desirably proximate the joint 302, the delivery device and/or the device 100′ are manipulated to unfurl or otherwise expand the device 100′. This “positioning” portion of the process is shown in FIG. 9B. The device can be delivered and manipulated by a laparoscopy device, through an open procedure, by robotic methods such as a da Vinci robotic surgical system, or by any other suitable methods or combinations of methods. The aforementioned encircling loop is formed (as shown in FIG. 9C) and any desired attachments are made between the device 100′, components thereof, and/or patient tissues, in any desired manner. (E.g, suitably configured adhesives could be activated.)

As shown in FIG. 9C, the device 100′ achieves and maintains the operative configuration for a desired amount of indwelling. During that maintenance of the device 100′ in the operative configuration, liquid (e.g, adhesive activator and/or healing substances such as pharmaceuticals or blood products) is permitted to penetrate through the membrane 114′ in a first direction D1 from the outer surface 212 toward the inner surface 108. In addition, liquid (e.g., urine) is substantially prevented from penetrating through the membrane 114′ in a second direction opposition the first direction.

Once the device 100′ is no longer desired to help seal and/or support the joint 302′ and related patient tissues, the device 100′ could biodegrade, could be removed in any desired manner, and/or could simply be allowed to remain undisturbed.

Though the description herein references first and second patient tissue structures 304 and 306 and a joint 302, it is contemplated that any desired number of patient tissue structures could be connected by a corresponding number of joints 302, with the assistance of any desired number of devices 100. One of ordinary skill in the art could readily configure a device 100 in any suitable substantially planar or three-dimensional shape to assist with sealing and/or supporting the joint(s) 302 as desired for a particular use environment.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. Fingers 534 could extend “downward” from the bottom band portion 432 also or instead of “upward” from the top band portion 430, as shown in the Figures. Installation of the device 100, 100′ could be accomplished by any type of surgical techniques, including laparoscopic, open, robotic, any other surgical access style/scheme, or any combination thereof. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. A device for supporting a joint formed by adjacent first and second patient tissue structures, the device comprising:
an inner surface of a device body, the inner surface being directly adjacent to the joint and attached to at least one of the first and second patient tissue structures during use; and
an outer surface of the device body, oppositely facing from the inner surface, the outer surface including a semi-permeable membrane, the membrane permitting liquid to penetrate therethrough in a first direction from the outer toward the inner surface, but the membrane substantially preventing liquid from penetrating therethrough in a second direction opposite the first direction.

2. The device of claim 1, wherein the inner layer directly contacts both of the first and second patient tissue structures during use.

3. The device of claim 1, including at least one reinforcement feature locally resisting force applied against the device in substantially at least one of the first and second directions.

4. The device of claim 3, wherein the reinforcement feature is an integral feature resulting from a thickness of a local portion of the device body.

5. The device of claim 3, wherein the reinforcement feature is a separate strut provided to the device.

6. The device of claim 5, wherein the strut is encapsulated within the device body.

7. The device of claim 5, wherein the strut is attached to the outer surface.

8. The device of claim 1, wherein the device body includes a band having a first band end and a second band end laterally separated by a band length, the first and second band ends being configured for selective mutual interconnection, and a top band portion and a bottom band portion longitudinally separated by a band height.

9. The device of claim 8, wherein the device body includes a plurality of fingers extending substantially longitudinally from the top band portion away from the bottom band portion.

10. The device of claim 1, wherein at least a portion of the inner surface includes an adhesive.

11. The device of claim 10, wherein the semi-permeable membrane allows an activator to permeate through the device body to activate the adhesive.

12. The device of claim 10, wherein the adhesive at least partially attaches the inner surface to at least one of the first and second patient tissue structures during use.

13. The device of claim 10, wherein the adhesive is at least one of a thermally-activated bioadhesive, a chemically-activated bioadhesive, a photo-activated bioadhesive, and a hydrogel glue.

14. The device of claim 1, including a coupling feature selectively attaching to a delivery device to aid placement of the device at/near the joint.

15. A method of supporting a joint formed by adjacent first and second patient tissue structures, the method comprising:
providing a device including:
an inner surface of a device body, and
an outer surface of the device body, oppositely facing from the inner surface, the outer surface including a semi-permeable membrane;
removably attaching the device to a delivery device;
with the delivery device, manipulating the device into proximity with the joint to establish an operative configuration such that the inner surface is directly adjacent to the joint in a supporting relationship and the inner surface is attached to at least one of the first and second patient tissue structures;
maintaining the device in the operative configuration;
permitting liquid to penetrate through the membrane in a first direction from the outer toward the inner surface; and
substantially preventing liquid from penetrating through the membrane in a second direction opposite the first direction.

16. The method of claim 15, wherein manipulating the device into proximity with the joint to establish an operative configuration such that the inner surface is directly adjacent to the joint and the inner surface is attached to at least one of the first and second patient tissue structures includes placing the inner layer into direct contact with at least one of the first and second patient tissue structures.

17. The method of claim 16, wherein placing the inner layer into direct contact with at least one of the first and second patient tissue structures includes adhering the inner layer to the at least one of the first and second patient tissue structures.

18. The method of claim 15, including locally resisting force applied against the device in substantially at least one of the first and second directions with at least one reinforcement feature.

19. The method of claim 18, wherein providing a device includes providing the reinforcement feature as an integral feature resulting from a thickness of a local portion of the device body.

20. The method of claim 19, wherein providing a device includes providing the reinforcement feature as a separate strut provided to the device.

21. The method of claim 20, wherein providing the separate strut to the device includes encapsulating the strut within the device body.

22. The method of claim 20, wherein providing the separate strut to the device includes attaching the strut to the outer surface.

23. The method of claim 15, wherein providing the device includes
including in the device body a band having a first band end and a second band end laterally separated by a band length, the first and second band ends being configured for selective mutual interconnection, and a top band portion and a bottom band portion longitudinally separated by a band height; and wherein
manipulating the device into proximity with the joint to establish an operative configuration includes
manipulating the band into an operative configuration of a circumferentially extending loop encircling at least a portion of the joint, and
with the band in the operative configuration, connecting the first band end and the second band end to maintain the band in the operative configuration.

24. The method of claim 23, wherein connecting the first band end and the second band end includes allowing an activator to permeate through the semi-permeable membrane to activate an adhesive on at least one of the first and second band ends to form the connection.

25. The method of claim 15, including sealing the joint with the device.

26. The method of claim 23, wherein supporting the joint includes providing the device body with a plurality of fingers extending substantially longitudinally from the top band portion away from the bottom band portion.

27. The method of 26, wherein locally resisting force applied against the device includes, with at least one reinforcement feature, locally resisting force applied against at least one of the plurality of fingers in substantially at least one of the first and second directions.

28. The method of claim 27, wherein connecting the first band end and the second band end to maintain the band in the operative configuration includes imparting a crown-like configuration to the device in an operative configuration.

29. A device for supporting a joint formed by adjacent first and second patient tissue structures, the device comprising:
a device body including a band having a first band end and a second band end laterally separated by a band length, the first and second band ends being configured for selective mutual interconnection, and a top band portion and a bottom band portion longitudinally separated by a band height;
an inner surface of the device body, the inner surface being directly adjacent to the joint and attached to at least one of the first and second patient tissue structures during use; and
an outer surface of the device body, oppositely facing from the inner surface, the outer surface including a semi-permeable membrane, the membrane permitting liquid to penetrate therethrough in a first direction from the outer toward the inner surface, but the membrane substantially preventing liquid from penetrating therethrough in a second direction opposite the first direction.

30. The device of claim 29, wherein the inner layer directly contacts both of the first and second patient tissue structures during use.

31. The device of claim 29, wherein the device body includes a plurality of fingers extending substantially longitudinally from the top band portion away from the bottom band portion.

32. The device of claim 31, including at least one reinforcement feature locally resisting force applied against at least one of the plurality of fingers in substantially at least one of the first and second directions.

33. The device of claim 32, wherein the reinforcement feature is an integral feature resulting from a thickness of at least a portion of the at least one of the plurality of fingers.

34. The device of claim 32, wherein the reinforcement feature is a separate strut provided to the device and attached to at least one of at least a portion of the at least one of the plurality of fingers and at least a portion of the band.

35. The device of claim 34, wherein the strut is encapsulated within the device body.

36. The device of claim 34, wherein the strut is attached to the outer surface.

37. The device of claim 31, wherein each of the plurality of fingers has length between about one centimeter (cm) and about 3 cm.

38. The device of claim 31, wherein each of the plurality of fingers has a length of about 1 millimeter (mm).

39. The device of claim 29, wherein at least a portion of the inner surface includes an adhesive.

40. The device of claim 39, wherein the semi-permeable membrane allows an activator to permeate through the device body to activate the adhesive.

41. The device of claim 39, wherein the adhesive at least partially attaches the inner surface to at least one of the first and second patient tissue structures during use.

42. The device of claim 29, wherein the first band end and the second band end are connected in an operative configuration to form a circumferentially extending loop encircling at least a portion of the joint.

43. The device of claim 31, wherein the device has a crown-like configuration in the operative configuration.

44. The device of claim 29, including a coupling feature selectively attaching to a delivery device to aid placement of the device at/near the joint.
